(19) United States
(12) Patent Application Publication
(10) Pub. No.: US 2021/0312942 A1
(43) Pub. Date: Oct. 7, 2021

(54) SYSTEM, METHOD, AND COMPUTER PROGRAM FOR COGNITIVE TRAINING

(71) Applicant: WINTERLIGHT LABS INC., Toronto (CA)

(72) Inventors: Frank RUDZICZ, Toronto (CA); Kathleen FRASER, Ottawa (CA); Liam KAUFMAN, Toronto (CA); Maria YANCHEVA, Toronto (CA)

(21) Appl. No.: 17/223,261

(22) Filed: Apr. 6, 2021

Related U.S. Application Data
(60) Provisional application No. 63/005,637, filed on Apr. 6, 2020.

(51) Int. Cl. G10L 25/66; G06N 3/08; G06N 3/04; G16H 20/70; G10L 25/24; G06F 40/284; G06F 40/30; G10L 15/18; G06F 40/211; G10L 15/22; G10L 15/02; A61B 5/00

(52) U.S. Cl. CPC G10L 25/66; G06N 3/08; G06N 3/0445; G16H 20/70; G10L 25/24; G06F 40/284; A61B 5/4803; G10L 15/1815; G06F 40/211; G10L 15/22; G10L 15/02; A61B 5/4088; G06F 40/30

(57) ABSTRACT

There is provided a system, method, and computer program for cognitive assessment scoring and planning in the field of neurological and/or behavioral testing. A system for automatic scoring for cognitive assessment can include quantifying cognitive states given automatic measures applied to language data. A system for automatic construction of assessment plans can include quantifying cognitive states using mathematical models, given measures extracted automatically from speech and language data.

### TECHNICAL FIELD

The following relates generally to automated and intelligent cognitive training.

### BACKGROUND OF THE INVENTION

Research into the early assessment of dementia is becoming increasingly more important, as the proportion of people affected by it grows every year. Changes in cognitive ability due to neurodegeneration associated with Alzheimer's disease (AD) lead to a progressive decline in memory and language quality. Assessment techniques include games to improve memory, a computer-based cognitive assessment system, and a psychological testing method. However, there can be various challenges and implementation problems with currently available alternatives.

It is therefore an object of the following to obviate or mitigate the above disadvantages.

### SUMMARY OF THE INVENTION

In one aspect, a system for scoring language tasks for assessment of cognition is provided, the system comprising: a collector configured to collect language data, the language data comprising at least one of speech, text, and a multiple-choice selection; an extractor configured to extract a plurality of language features from the collected language data using an automated language processing algorithm, the plurality of language features comprising at least one of an acoustic measure, a lexicosyntactic measure, and a semantic measure; and a score producer configured to use the extracted plurality of language features to automatically produce a plurality of scores, the plurality of scores generated using an automated language processing algorithm.

In another aspect, a system for constructing a plan for assessment of cognition is provided, the system comprising: a dictionary comprising a plurality of tasks; a task profile set comprising a task profile for each of the plurality of tasks; a user profile based at least in part on a user's prior performance of a subset of the plurality of tasks; a target metric; and a plan constructor configured to conduct an analysis of the dictionary, the task profile set, and the user profile, and to select and order one or more of the plurality of tasks to optimize the target metric based at least in part on the analysis.

In yet another aspect, a method of dynamically determining a next task in a cognitive assessment is provided, the method comprising: obtaining one or more performance measurements of a first task; approximating a clinical score from the one or more performance measurements of the first task; inputting the clinical score into an expectation-maximization function; obtaining a score approximation from the expectation-maximization function; generating a first parameter based on the score approximation and a target metric; identifying one or more candidate tasks based on the first parameter and the target metric; for each of the one or more candidate tasks, calculating a reward score based on the candidate task and the first parameter; generating a second parameter based on the reward score and the first parameter; and selecting the next task from the one or more candidate tasks that maximizes the target metric.

### BRIEF DESCRIPTION OF THE DRAWINGS

A greater understanding of the embodiments will be had with reference to the figures, in which:

FIG. 1 illustrates a block diagram of a system for cognitive assessment scoring and planning, according to an embodiment.

FIG. 2 illustrates a flow diagram of a method of scoring language tasks for assessment of cognition, according to an embodiment.

FIG. 3 illustrates a block diagram of an exemplary system for scoring language tasks for assessment of cognition, in accordance with the system of FIG. 1.

FIG. 4 illustrates a flow diagram of a method of automating the process to regress on unknown output variables given measurements, according to an embodiment.

FIG. 5A illustrates a block diagram of an exemplary automated system for constructing an assessment plan for neurological and/or behavioral testing, in accordance with the system of FIG. 1.

FIG. 5B illustrates a block diagram of exemplary components of the plan constructor of FIG. 5A.

FIG. 6 illustrates a flow diagram of a method of constructing an assessment plan for neurological and/or behavioral testing, according to an embodiment.

FIG. 7 illustrates a flow diagram of a method of dynamically determining a next task in a cognitive assessment, according to an embodiment.

### DETAILED DESCRIPTION OF VARIOUS EMBODIMENTS OF THE INVENTION

Embodiments will now be described with reference to the figures. For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures, and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

Any module, unit, component, server, computer, terminal, engine, or device exemplified herein that executes instructions may include or otherwise have access to computer-readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer-readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information, and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application, or module herein described may be implemented using computer readable/executable instructions that may be stored or otherwise held by such computer-readable media and executed by the one or more processors.

Alzheimer's disease (AD) and dementia generally cause a decline in memory and language quality. Patients typically experience deterioration in sensory, working, declarative, and non-declarative memory, which leads to a decrease in the grammatical complexity and lexical content of their speech. Current methods for identification of AD include costly and time-consuming clinical assessments with a trained neuropsychologist who administers a test of cognitive ability, such as the Mini-Mental State Examination (MMSE), the Montreal Cognitive Assessment (MoCA), and the Repeatable Battery for the Assessment of Neuropsychological Status (RBANS). These clinical assessments include many language-based questions which measure language production and comprehension skills, speech quality, language complexity, as well as short-term recall, attention, and executive function.

Cognitive and motor assessments often involve the performance of a series of tasks. For instance, the MMSE, a standard assessment of cognition, involves a short, predetermined series of subtasks including ‘orientation’, followed by ‘registration’, ‘attention’, ‘recall’, and ‘language’ in succession. Typically, assessments contain a single or a small number of versions of each subtask, each being of approximately the same level of difficulty. Moreover, different assessment task types have historically been designed to evaluate different aspects of cognitive function. For example, the Stroop test is useful in evaluating executive function (i.e., the ability to concentrate on a task in the presence of distractors), picture-naming is a simple elicitor of word recall, and question-answering is a simple elicitor of semantic comprehension.

The present disclosure provides systems, methods, and computer programs providing self-administered cognition assessment. Embodiments generally provide technological solutions to the technical problems related to automating self-administered computer-based assessment of cognition and constructing a plan for computer-based assessment of cognition. Automating self-administered computer-based assessment of cognition poses the technical challenge of using a computer to more effectively interact with the subject than could an expert, automate a scoring process so that the subject does not need to interact with a person, and utilize aggregate data in a seamless manner in real time. Constructing a plan for computer-based assessment of cognition poses the technical challenge of using a computer to dynamically optimize constituent tasks and task instances, reduce the quantity of human-computer interaction while improving precision of cognitive assessment, and improve the accuracy of cognitive assessment when a particular task score produces an ambiguous symptom output.

These embodiments can be more generally applied across pathologies and are sensitive to the differences between very similar pathologies. For example, Parkinson's disease and Lewy body dementia have very similar presentations in terms of muscle rigidity, but the latter is more commonly associated with delusion, hallucination, and memory loss (which itself may appear similarly to Alzheimer's disease). In order to isolate hallucination from muscle rigidity and memory loss, for example, appropriate tasks need to be assigned as it may be impractical and time-consuming to perform a full battery of tests. Furthermore, the described embodiments enable a dynamic variation of task difficulty, which is adjusted according to the performance of each participant, in order to capture fine-grained cognitive issues in early-, moderate-, and late-stage impairment.

One of the objectives of the described embodiments is to provide a system capable of producing an assessment plan (i.e., a series of tasks with specific stimuli instantiations) based on a numeric score that is computed by a possible combination of quantifiable goals. One exemplary goal is identifying single dimensions of assessment that require greater resolution (e.g., if insufficient statistics are computed on grammatical complexity, more tests for grammatical complexity should be assigned). Another exemplary goal is identifying pairs of dimensions that would offer discriminable information for a classification decision (e.g., if the system could not diagnose between Parkinson's and Lewy-body dementia, more tests for memory loss would be assigned).

The described embodiments may be configured to overcome various shortcomings in manual processes, memory-improvement games, computer-based cognitive assessment systems, and psychological testing methods.

One such shortcoming is the rigid task order of the foregoing approaches. If an individual's challenges are concentrated in one area, such as language, having a rigid assessment order and rigid proportion of subtasks in each area of assessment would not flexibly focus in on the areas with greatest salience for diagnosis. The effect of directed attention fatigue can also be an issue and distribute performance unevenly across an assessment.

Another shortcoming is uniform task difficulty. When assessments are administered on participants with varying cognitive levels, a single level of difficulty is inappropriate for all participants. If the uniform difficulty is too low, a cognitively healthy individual will perform well on all subtasks, leading to a ‘ceiling effect’ where the scores of the assessment are not informative. Conversely, if the difficulty is too high, a cognitively impaired individual will perform poorly on all subtasks, leading to a ‘floor effect’. This renders assessments either too coarse for assessment of mild cognitive impairment (e.g., the MMSE) or too difficult for assessment of late-stage impairment (e.g., the MoCA).

Other potential shortcomings include: (a) no history or incorporation of longitudinal information; (b) no stress level or sentiment analysis; (c) a lengthy process in which a patient might get tired and thus start to perform worse; (d) the need for a user to create an account in order to access results data; and (e) visual feedback from getting incorrect answers might stress out the user and lead to more incorrect answers.

The described embodiments automatically assess language tasks to conduct more efficient and timely assessments. These efficiencies may be achieved by providing self-administration of the language tasks through a responsive computer-based interface; enabling longitudinal assessments and preventing a ‘learning effect’ over time through the use of a large bank of automatically generated task instances; and automatically generating scores for a battery of language tasks. These consequently may enable frequent monitoring of cognitive status in elderly adults, even before symptoms of dementia become apparent. Early identification of the preclinical stages of the disease would be beneficial for studying disease pathology and enabling researchers to test disease-modifying therapies.

In one aspect, there is provided a system, method, and computer program for automated scoring of language tasks for assessment of cognition. In an embodiment, the system collects language data, the language data including speech, text, and/or a multiple-choice selection. The system extracts language features from the collected language data using automated language processing. In embodiments, the language features include an acoustic measure, a lexicosyntactic measure, and/or a semantic measure. The system uses the extracted language features to automatically produce scores, the scores generated using the automated language processing. The scores may subsequently be used for assessment planning.

In another aspect, there is provided a system, method, and computer program for constructing a plan for assessment of cognition. In an embodiment, the system has or is capable of receiving a dictionary of tasks. The system creates or is capable of receiving a set of task profiles for each of the tasks. The system creates or is capable of receiving a user profile based at least in part on a user's prior performance of a subset of the tasks. The system generates a target metric, or it allows for a target metric to be input by a user or an external source. The system conducts an analysis of the dictionary, the task profile set, and the user profile; using this analysis, the system selects and orders one or more tasks to optimize the target metric.

In another aspect, a method of scoring language tasks for assessment of cognition may be combined with a method of automatically constructing an assessment plan. In so doing, the parameters that could be learned independently by each method can be learned simultaneously, e.g., using expectation-maximization. A computer implementation of such a combination of these parts enables the dynamic determination of a next task in a cognitive assessment by performing a number of steps, for example, in sequence, concurrently, or both. A system may be configured to perform these steps, which may include: obtaining one or more performance measurements of a first task; approximating a clinical score from the one or more measurements of task performance; inputting the clinical score in an expectation-maximization function; obtaining score approximation from the expectation-maximization function; generating a first parameter based on the score approximation; determining the next task based on the first parameter; calculating a reward score based on the next task and the first parameter; generating a second parameter based on the reward score and the second parameter; and presenting the next task.

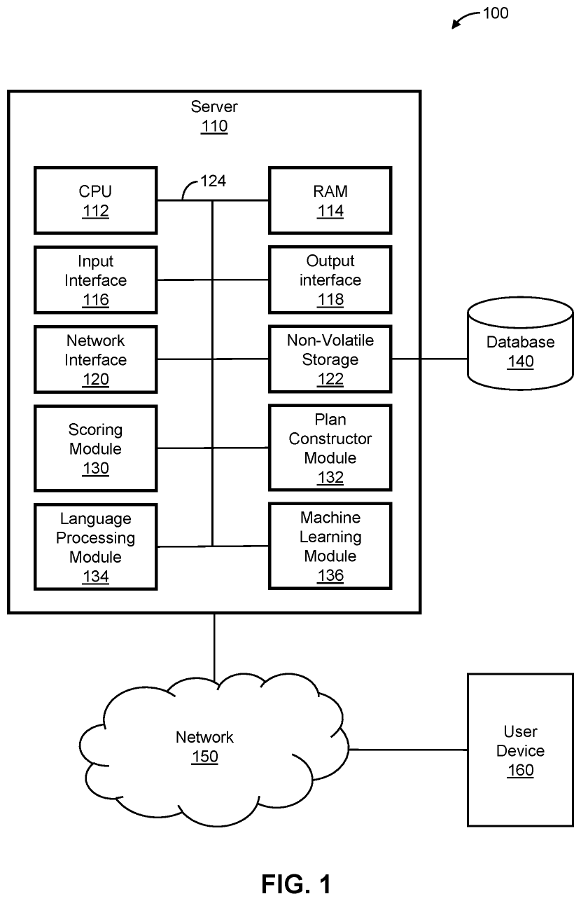

Referring now to FIG. 1, a system for cognitive assessment scoring and planning 100, in accordance with an embodiment, is shown. The system 100 generally comprises a server 110 and a user device 160 communicatively linked to the server 110 by a network 150 (such as the Internet). The server 110 implements assessment scoring and planning, while the user device 160 provides a user interface for enabling a subject to undergo cognitive assessment as directed by the server 110.

FIG. 1 shows various physical and logical components of an embodiment of system 100. As shown, server 110 has a number of physical and logical components, including a central processing unit (“CPU”) 112 (comprising one or more processors), random access memory (“RAM”) 114, an input interface 116, an output interface 118, a network interface 120, non-volatile storage 122, and a local bus 124 enabling CPU 112 to communicate with the other components. CPU 112 executes an operating system, and various modules, as described below in greater detail. RAM 114 provides relatively responsive volatile storage to CPU 112. Input interface 116 enables an administrator or user to provide input via an input device, such as a keyboard, touchscreen, or microphone. Output interface 118 outputs information to output devices, such as a display and/or speakers. In some cases, input interface 116 and output interface 118 can be the same device (e.g., a touchscreen or tablet computer). Network interface 120 permits communication with other systems, such as user device 160 and servers remotely located from the server 110, such as for a typical cloud-based access model. Non-volatile storage 122 stores the operating system and programs, including computer-executable instructions for implementing the operating system and modules, as well as any data used by these services. Additional stored data, as described below, can be stored in a database 140. Database 140 may be local (e.g., coupled to server 110). In other embodiments, database 140 may be remote (e.g., accessible via a web server). Data from database 140 may be transferred to non-volatile storage 122 prior to or during operation of the server 110. Similarly, data from non-volatile storage 122 may be transferred to database 140. During operation of the server 110, the operating system, the modules, and the related data may be retrieved from the non-volatile storage 122 and placed in RAM 114 to facilitate execution. In some embodiments, the server 110 further includes a scoring module 130, a plan constructor module 132, a language processing module 134, and/or a machine learning module 136. In some embodiments, user device 160 runs an application that allows it to communicate with server 110 remotely. In other embodiments, server 110 may also be the user device 160 in a standalone application that need not communicate with a network (such as the Internet).

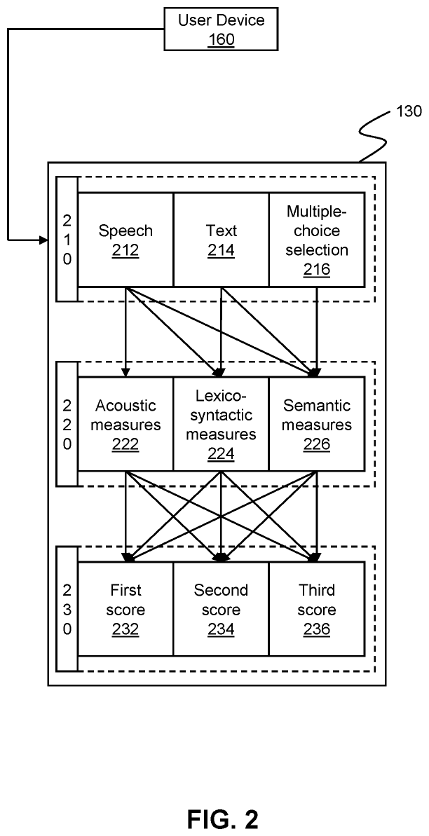

FIG. 2 illustrates a block diagram of exemplary components of the scoring module 130 of FIG. 1. A collector 210 collects language data, including, but not limited to, speech 212, text 214, and multiple-choice selection 216, that was generated by a subject on user device 160.

An extractor 220 extracts language features from the collected data using automated language processing techniques. The features extracted by the extractor 220 include acoustic measures 222, lexicosyntactic measures 224, and semantic measures 226. Acoustic measures 222 are extracted from the verbal responses to obtain Mel-frequency cepstral coefficients (MFCCs), jitter and shimmer measures, aperiodicity features, measures of signal-to-noise ratio, pauses, fillers, and features related to the pitch and formants of the speech signal. Lexicosyntactic measures 224 are extracted from textual responses and transcriptions of verbal responses, and include frequency of production rules, phrase types, and word types; length measures; frequency of use of passive voice and subordination/coordination; and syntactic complexity. Semantic measures 226 are extracted by comparing subject responses to ground truth (i.e., expected) responses to each task, such as dictionary definitions for a given word or thematic units contained in a given picture.

A score producer 230 uses the extracted language features to automatically produce scores, such as a first score 232, a second score 234, and a third score 236, for every type of language task, which can be used as a substitute for, or in addition to, the manually produced clinical scores for the task. The scores may, but need not, correspond to specific extracted language features. The automatic scores produced by the score producer 230 are generated using language processing algorithms, such as, but not limited to, models for semantic similarity among words or larger passages, computation of distance between vector representations of words or larger passages, traversal of graph-based representations of lexical and linguistic relations, computation of lexical cohesiveness and coherence, topic identification, and summarizing techniques.

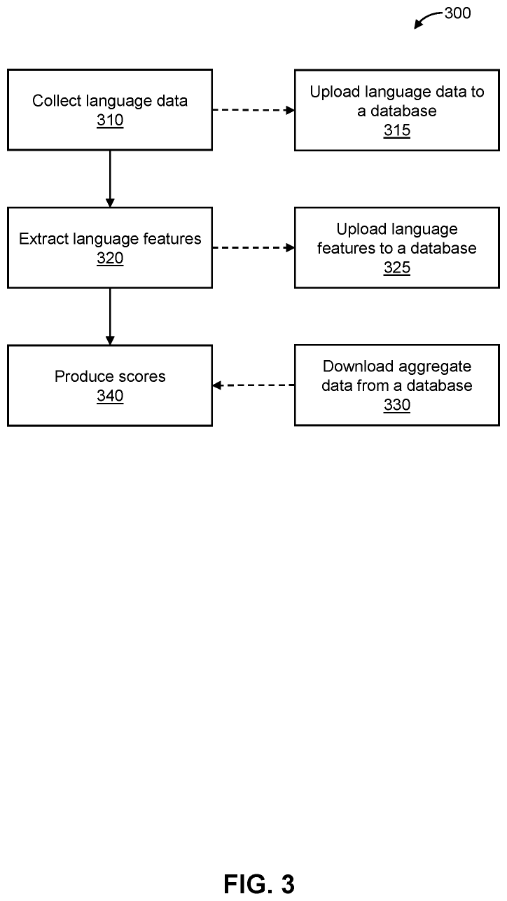

FIG. 3 illustrates an automated method for scoring language tasks for assessment of cognition 300, in accordance with an embodiment. Language tasks to be scored may include vocabulary assessment through word definition, image naming, picture description, sentence completion/re-ordering, story recall, Winograd schema problems, phrase re-ordering, random item generation, color naming with Stroop interference, and self-assessed general disposition.

At block 310, language processing module 134 collects language data (including, but not limited to, speech, text, multiple-choice selection, touch, gestures, and/or other user input) generated by a subject. Optionally, the language data may have been stored on database 140 from a previous session, and language processing module 134 collects language data from database 140. At block 315, CPU 112 may upload the language data to database 140.

At block 320, language processing module 134 extracts language features from the collected data using automated language processing algorithms. At block 325, language processing module 134 may upload the language features to database 140. The language features may include acoustic, lexicosyntactic, and semantic measures. Acoustic measures may be extracted from the verbal responses to obtain Mel-frequency cepstral coefficients (MFCCs), jitter and shimmer measures, aperiodicity features, measures of signal-to-noise ratio, pauses, fillers, and features related to the pitch and formants of the speech signal. Lexicosyntactic measures may be extracted from textual responses and transcriptions of verbal responses, and may include frequency of production rules, phrase types, and word types; length measures; frequency of use of passive voice and subordination/coordination; and syntactic complexity. Semantic measures may be extracted by comparing subject responses to ground truth (i.e., expected) responses to each task, such as dictionary definitions for a given word or thematic units contained in a given picture.

At block 330, language processing module 134 may download aggregate data comprising language data and language features from database 140.

At block 340, language processing module 134 uses the extracted language features to automatically produce scores for every type of language task, which can be used as a substitute for, or in addition to, the manually produced clinical scores for the task. Language processing module 134 may also use some or all of the aggregate data from database 140 as part of the input to produce scores. The scores may be generated using language processing algorithms, such as, but not limited to, models for semantic similarity among words or larger passages, computation of distance between vector representations of words or larger passages, traversal of graph-based representations of lexical and linguistic relations, computation of lexical cohesiveness and coherence, topic identification, and summarizing techniques. In addition to producing scores, a confidence value for each score may be generated based on some or all of the collected language data and/or some or all of the extracted language features.

Method 300 may be implemented on a web-based application residing on user device 160 that communicates with a server 110 that is accessible via the Internet through network 150. Multiple other subjects may use the same web-based application on their respective user devices to communicate with server 110 to take advantage of aggregate data. In such a case, some or all of the user devices of the multiple other subjects may automatically upload collected language data to server 110. Similarly, the user devices of the multiple other subjects may automatically upload extracted language features to server 110. This aggregate data would then reside on server 110 and be accessible by the web-based application used by the multiple other subjects. Each web-based application can then determine a ‘ground truth’ based on this aggregate data.

The ground truth is an unambiguous score extracted from validated procedures. The ground truth can include such measures as a count, an arithmetic mean, or a sum of boxes measure. The types of ground truths that may be generated or used can depend on the task and on what the medical community has decided by consensus. For example, in an animal naming task, the number of items named can be used, but one might subtract blatantly incorrect answers from the score. For example, for a picture description task, an arithmetic combination of total utterances, empty utterances, subclausal utterances, single-clause utterances, multi-clause utterances, agrammatic deletions, and a complexity index can be combined into a ground truth. The total number of information units mentioned can also provide a ground truth in picture description.

For example, there might be a first task requiring subjects to name all the animals they can think of and a second task requiring them to describe a picture. Here, the number of animals they name can be used as an anchor if it is considered a good indicator of performance. The ‘goodness’ of an indicator variable can be devised by whether the measure is validated. In this same example, if the computation of ground truth is an unambiguous measure from the scientific literature, that would be used. The validation may be programmed into the system prior to use (e.g., based on the scientific literature), dynamically (e.g., based on changing answers obtained from users of the system), or both. In a particular case, the system can rely on the literature and the scientific consensus first. In another cases, the system can rely on analysis of the received data; e.g., in picture descriptions, information units can be useful, even if they do not appear in previously studied rating scales.

The subjects may then be ranked according to performance on this first task. Principal components analysis (PCA), or another dimensionality reduction technique, can then be used on each dimension (e.g., measured performance) to determine which factors (i.e., aggregate of features) are important in scoring individual subjects. In addition, plan constructor module 132 can use the PCA as data for constructing a plan for assessment of cognition.

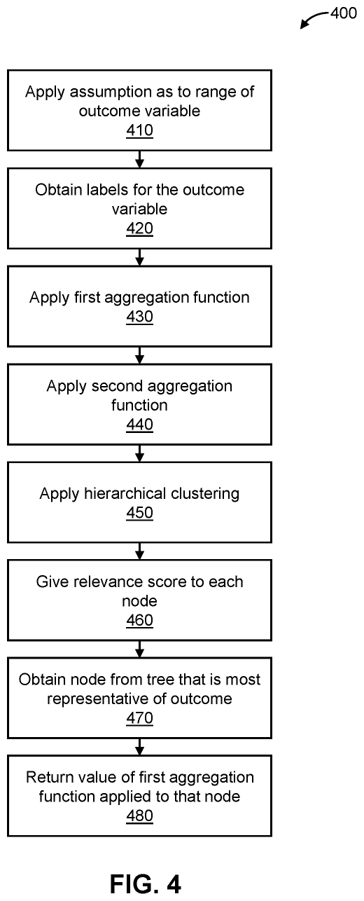

FIG. 4 illustrates a method of regression on unknown output variables given measurements 400, in accordance with an embodiment. Machine learning module 136 is configured to provide an automatic process that takes a set of features and sub-scores to generate a single outcome measure for use by score producer 230. The process may operate on a set X of features {x₁, x₂, . . . , xₙ}, a set Y of interpretable sub-scores {y₁, y₂, . . . , yₘ} (e.g., word-finding difficulty, hypernasality, etc.), a single outcome measure O, and human interpreters I={I₁, I₂, . . . , I_{K}}.

At block 410, machine learning module 136 applies an assumption as to the range of an outcome variable. More specifically, machine learning module 136 may apply an assumption as to the range of the outcome variable O, and/or the sub-scores in Y. For example, machine learning module 136 may assume that 0 and Y are continuous on [0 . . . 1], but other scales may also be applied. Furthermore, different scales for different sub-scores may be applied.

At block 420, machine learning module 136 obtains labels for the outcome variable from a subset of human interpreters. More specifically, machine learning module 136 may obtain labels lᵢ∈{−,+} for O from a subset of human interpreters for each variable of X, where a label indicates whether or not the given feature xᵢ is negatively or positively related with the outcome O. A lack of a label does not necessarily indicate no relation. In another embodiment, these labels can be more fine-grained on a Likert-like scale (e.g., indicating degree of relation). In yet another embodiment, these labels are not applied to outcome variable O but to some subset of sub-scores in Y.

At block 430, machine learning module 136 applies a first aggregation function that provides scores based on the relationship between features and labels. More specifically, machine learning module 136 may apply an aggregation function αₓ(xᵢ,lᵢ) that provides higher scores when xᵢ∈X and lᵢ are highly related and lower scores when they are inversely related. Examples of the aggregation function include degrees of correlation (e.g., Spearman) and mutual information between the provided arguments. The function α may only be computed over the subset of instances for which a label exists. The function α may first aggregate labels across interpreters I for each datum; for example, the mode of labels may be taken.

At block 440, machine learning module 136 applies a second aggregation function to pairs of features regardless of the presence of labels. More specifically, machine learning module 136 may apply an aggregation function β(xᵢ,xⱼ) to pairs of features xᵢ,xⱼ ∈X regardless of the presence of labels. This reveals pairwise interactions between all features. Examples of the aggregation function include degrees of correlation (e.g., Spearman) and mutual information between the provided arguments.

At block 450, machine learning module 136 applies hierarchical clustering to obtain a graph structure over all features; in this case, a tree structure using the second aggregation function as a distance metric. In other cases, other graph structures, such as tree-like structures, can be used. For this case, more specifically, machine learning module 136 may, using β(nᵢ,nⱼ) as the distance metric, apply hierarchical clustering (either bottom-up or top-down) to obtain a tree structure over all features. The arguments of β are generally the nodes representing aggregates of its subsumed components. The resulting tree structure represents an organization of the raw features and their interconnections. Data constituting the arguments of β can be arbitrarily aggregated. For example, if nᵢ is the aggregate of features x₁ and x₂, all values of x₁ and x₂ can be concatenated together, or they can be averaged.

At block 460, machine learning module 136 gives a relevance score to each node within the tree, using the first aggregation function as a relevance metric. More specifically, using αₙ(nᵢ,n₁) as the relevance metric, each node within the tree produced at block 450 may be given a relevance score. For example, if x₁ and x₂ are combined into node nᵢ according to block 450, the relevance score of node nᵢ may be:
the average of α(x₁,l) and α(x₂,l);
the sum of α(x₁,l) and α(x₂,l); or
λ·α(x₁,l)+(1−λ)·+(x₂,l), where λ∈[0 . . . 1] and may be determined by a variety of methods including, e.g., the proportion of the variance in (x₁,x₂) explained by x₁ alone.

At block 470, machine learning module 136 obtains the node from the tree that is most representative of the outcome variable. More specifically, machine learning module 136 may, using an arbitrary function τ, obtain the node from the tree produced in block 450 that is most representative of outcome 0 or subscore Y. This may be done by first sorting nodes according to relevance scores obtained in block 460 and selecting the top-ranking node. This may also involve a threshold of relevance whereby if no score exceeds the threshold, no relationship is obtained.

At block 480, machine learning module 136 returns the value of the first aggregation function as applied to the node obtained from block 470. More specifically, the value of αₙ(nᵢ,nⱼ) may effectively become the outcome measure that would normally be obtained by regression, if there was such labeled data.

Although the foregoing description of exemplary method 400 provides eight blocks in which calculations may be performed, it will be appreciated that variations of the method with fewer blocks may be used. As an example, step 430 or 440 can be omitted. Hierarchical clustering at 450 can be replaced with another clustering method. Relevance scores may be replaced by some other ranking in step 460.

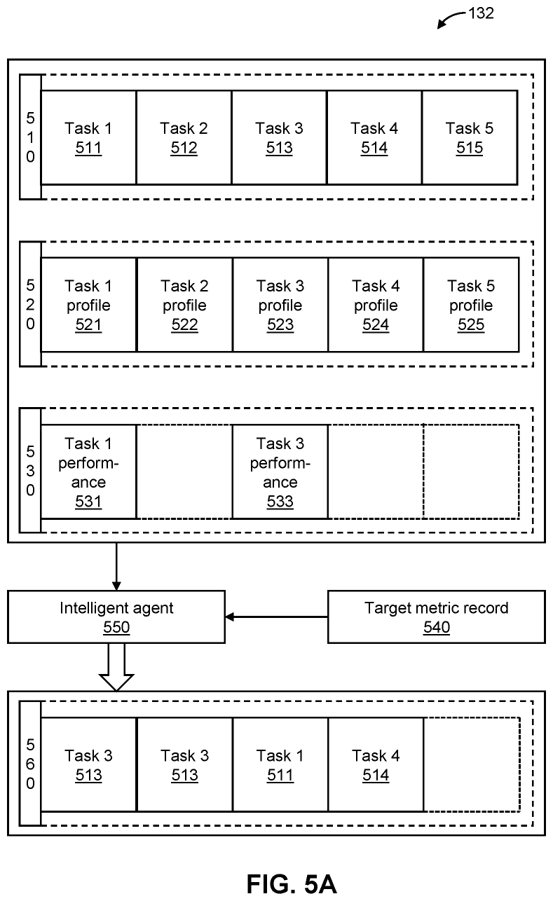

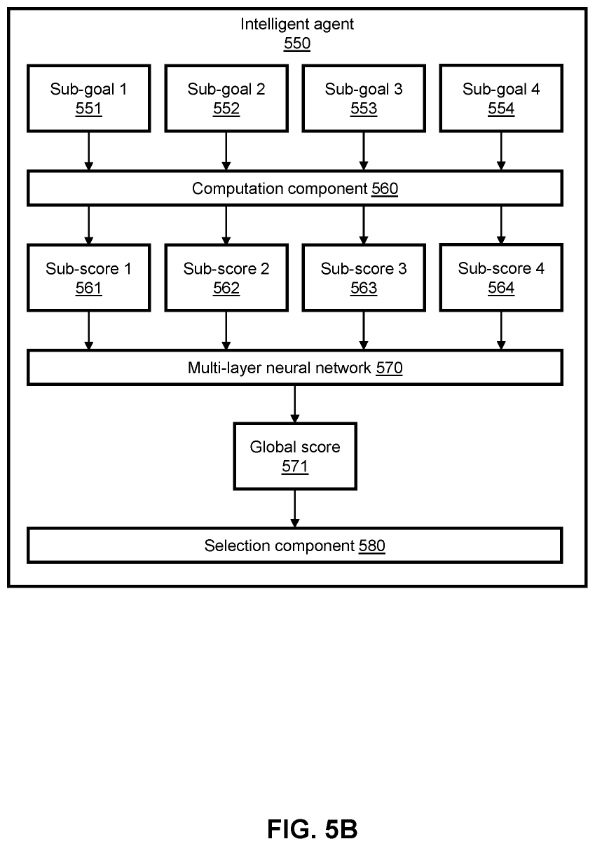

FIG. 5A illustrates a block diagram of exemplary components of the plan constructor module 132. Plan constructor module 132 automatically constructs an assessment plan for neurological and/or behavioral testing. Plan constructor module 132 comprises a dictionary 510, a task profile set 520, a user profile 530, a target metric record 540, and an intelligent agent 550. Plan constructor module 132 may be configured to automatically determine the next task in a sequence given a history of previous tasks and optionally a specified reward function.

In the embodiment shown in FIG. 5A, dictionary 510 is a dictionary or structure of available tasks, which may include task 1 511, task 2 512, task 3 513, task 4 514, task 515, and so on. For the purposes of illustration, five tasks are shown in this embodiment, but any suitable number of tasks may be provided in practice. A task is an activity that can be instantiated by various specific stimuli, and for which instructions for completion are explicitly given. Explicit scoring functions must also be given to each task. Tasks may include Stroop, picture naming, picture description, semantic or phonemic fluency, and the like.

A task profile set 520 is a set of profiles for each task, in terms of what aspects of assessment it explores (e.g., the picture-naming task projects onto the dimensions of semantic memory, vision, word-finding, etc.) and its difficulty level across those aspects. In this embodiment, task profile set 520 comprises five profiles, namely task 1 profile 521, task 2 profile 522, task 3 profile 523, task 4 profile 524, and task 5 profile 525. The aspects of assessment explored represent nominal categories, and the range of difficulty levels are on continuous, but otherwise arbitrarily sized, scales. Advantageously, each task and its difficulty levels assess more than one cognitive domain (as language is tied to memory and executive function). The tasks can also tease apart cognitive impairment, as compared to training a cognitive domain.

A user profile 530 is a profile of the user of the system, typically the subject being assessed, in terms of their prior performance on a subset of those tasks. In this embodiment, for illustration purposes, this subset consists of task 1 511 and task 3 513. User profile 530 accordingly comprises two performance records, here task 1 performance 531 and task 3 performance 533. Optionally, user profile 530 may also include demographic information. User profile 530 may include the raw scores obtained on previous tasks, and statistical models aggregating those scores.

A target metric record 540 stores a metric to optimize, supplied by a tester/clinician or by a virtual tester/clinician (e.g., developed through machine learning to replicate the decision-making done by a real tester/clinician). For example, a clinician might indicate that they are interested in exploring tasks that the subject completes with low accuracy (in order to better characterize the nature of the impairment). Alternatively, the clinician may want to maximize the precision of a diagnosis, by choosing tasks which are specifically related to a given set of diagnostic criteria.

Target metric record 540 may store a metric that has one or more of characteristics. Target metric record 540 may also store a combination of several metrics, for example, through a linear combination of scores, weighted by coefficients learnable from data or specified a priori. Target metric record 540 may be a function of user profile 530, so that the task and the stimulus within that task are selected to be within (or in accordance with) the abilities of the subject. Target metric record 540 may be a function of other metadata related to the interaction. For example, it may optimize engagement with the platform through longer sessions. This may involve aspects of sentiment. The arousal/valence/dominance model can be used, or elements from ‘gamification’. In some situations, the subject should not be so engaged that they use the system too much. In clinical settings, it is typical to avoid the practice effect.

Intelligent agent 550 is an intelligent computer agent that constructs a test plan 560, i.e., uses the four above sources of information to produce a sequence of tasks meant to optimize the target metric stored in target metric record 540. For the purposes of illustration, the intelligent agent 550 is shown to have produced a sequence of four tasks (repetition of tasks being allowed)—task 3 513, task 3 513, task 1 511, and task 4 514—that would constitute the test plan 560 to be presented to the subject.

One implementation of intelligent agent 550 would be a partially observable Markov decision process (POM DP) in which observations are data obtained through the use of the tool, the state is an assessment which is a portion of user profile 530, the reward/cost is related to target metric record 540, and the action is a list (or tree/graph) of tasks chosen from dictionary 510. Specifically, states can be inferred from sub-task scores, projections of feature vectors into factors, or other latent variables obtained through learning methods such as expectation-maximization.

In test plan 560, task instances can be repeated or selected without replacement up to arbitrary thresholds of recurrence. For example, a single task can be repeated continuously, only across sessions, only until all tasks within a task group are exhausted, only after some period of time has elapsed, or any other combination.

In addition, optionally, test plan 560 (which are structures of task instances created by the software program) presented to the subject can be lists, graphs, or other structures of tasks. A type of graph structure that can be used are tree or tree-like structures. For example, a ‘tree of tasks’ constitutes a decision tree in which one branch or another is followed, depending on the performance of the participant. Performance can be determined either deterministically or stochastically, e.g., through item response theory.

In addition, optionally, test plan 560 can be generated one-task-instance-at-a-time (thus accounting for subject's testing ability, given their current state of mental and/or physical health), all in advance (e.g., in a research setting), or constructed out of non-atomic subparts. Test plan 560 can also be edited dynamically (during use) by the software. This level of flexibility allows the examiner (clinician, caregiver, researcher) or subject (in case of self-administration) to administer cognitive assessment as appropriate given the subject's history and current condition (mental, physical, cognitive).

In other embodiments, intelligent agent 550 may allow for incorporating changes over time—personalize based on (1) current session and (2) longitudinal history. Intelligent agent 550 may also perform differential diagnostics or infer neuropsychological tests. The addition of these functionalities to intelligent agent 550 may be done to achieve the following objectives: (1) producing fine-grained diagnostic information (no ceiling/floor effect); and/or (2) reducing stress levels on subjects, including in cognitively impaired populations/errorless learning.

FIG. 5B illustrates a block diagram of exemplary components of the intelligent agent 550 of FIG. 5A. Intelligent agent 550 may construct an assessment plan which dynamically optimizes constituent tasks and task instances. Intelligent agent 550 takes as input any combination of the following sub-goals: (1) sub-goal 1 551 is to improve the extent of coverage; (2) sub-goal 2 552 is to improve the resolution of assessment; (3) sub-goal 3 553 is to improve the accuracy of assessment; and (4) sub-goal 4 554 is to reduce stress of the examinee.

Sub-goal 1 551 is to improve the extent/coverage of assessment by increasing scope in specific areas of difficulty or areas of ease for each subject. In typical assessments of cognition, such as the Mini-Mental State Examination (MMSE) or the Montreal Cognitive Assessment (MoCA), all tasks and task versions are fixed. When such assessments are administered to subjects of variable cognitive ability, a ‘ceiling effect’ may occur if the task instances are too easy for the subject, thereby resulting in perfect scores on all tasks. Conversely, a ‘floor effect’ may occur if the task instances are too difficult for the subject, resulting in low scores on all tasks. Such outcomes are not informative since they do not provide an indication of the extent of the subject's cognitive performance, when that performance falls outside of the range captured by the fixed set of tasks. Additionally, cognitive impairment may be heterogeneous across subjects. For instance, one subject may suffer from a syntax-related language impairment while another may experience visuospatial difficulties. While standard assessments of cognition consist of a fixed set of tasks, an assessment plan constructed by the method described above selects the tasks which are most relevant to the subject's specific impairment. As a result, assessment precision is improved in areas of interest to clinicians, and time spent on uninformative tasks is minimized.

Sub-goal 2 552 is to improve the resolution of assessment by increasing the statistical power in specific sub-areas of evaluation.

Sub-goal 3 553 is to improve the accuracy of assessment by improving differential diagnosis. Since many disorders present similar cognitive, behavioral, psychiatric, or motor symptoms, the assessment plan will dynamically select subsequent tasks and task instances which focus on resolving ambiguous symptoms. For instance, if a subject performs poorly on an image naming task, the word-finding difficulty could be caused by various disorders, including Lewy body dementia and major depression. In order to resolve the ambiguity, the assessment plan will select subsequent category-specific instances of the image naming task—if the anomia is observed to be specific to the category of living things, then it is more likely to be caused by Lewy body dementia than by depression.

Sub-goal 4 554 is to reduce stress and anxiety experienced by subjects who are completing the assessment.

A computation component 560 computes scalar ‘sub-scores’ for each of any combination of the above four sub-goals on any subset of the available tasks-stimuli instantiations. This produces, for example, four sub-scores 561, 562, 563, and 564. In this embodiment, a multi-layer neural network 570 combines the sub-scores into a single global score 571 derived from automatic analysis of data. The neural network at block 570 could be a ‘recurrent’ neural network or a neural network with an ‘attention mechanism’. Additionally, in the case where multiple instances are read, the components of intelligent agent 550 up to the neural network 570 could be replicated in sequence and fed into the single global score 571.

The data analyzed can include a combination of raw data, variables, and aggregate scores. The variables can include features (e.g., acoustic measures, such as MFCCs, jitter and shimmer measures, etc.) and interpretable sub-scores (e.g., word-finding difficulty, hypernasality). In other embodiments, the multi-layer neural network may produce weighted sub-scores in place of, or in addition to, the global score.

Computation component 560 may relate the sub-scores it calculates to the sub-goals discussed above. For example, a simple power analysis may be computed on task-stimuli instantiation X for sub-goal 2 552 (increasing statistical power of the latent aspects inferred by X). Each of these sub-scores may be normalized by any method, and on any scale (e.g., using z-score normalization).

Optionally, computation component 560 selects which tasks-stimuli instantiations require sub-scores. In some implementations, there are a tractable number of task-stimuli instantiations, but this module extends to scenarios where (a) there are too many task-stimuli pairs for which to compute all sub-scores quickly, or (b) there exist ‘dynamically created’ task-instantiation pairs.

The sub-scores calculated by computation component 560 may be combined into a single global score 571, denoted below as ‘g’, by any linear or non-linear combination of sub-scores. For example, for sub-score sᵢ and scalar coefficients cᵢ,

g=Σᵢcᵢsᵢ (1)

would constitute a linear computation of the single global score 571, and multi-layer neural network 570 combining inputs sᵢ would constitute a non-linear combination, where the coefficients cᵢ in the former and the various weights in the latter would be optimized from automatic analysis of data.

A selection component 580 selects task-stimuli instantiations from global score 571, as shown in this embodiment. Selection component 580 may, for example, iterate over all task-stimuli instantiations to create a list of these instantiations satisfying a particular condition based on the global score 571. In other embodiments, selection component 580 may use weighted sub-scores in place of, or in addition to, the global score 571 for the purposes of selecting task-stimuli instantiations.

Selection component 580 may select task-stimuli instantiations given either sub-scores, global scores, or both. This can be as simple as a list of these instantiations sorted by global score, or a more complex selection process that itself may be optimized from machine learning. For example, every instantiation type may be associated with global scores. These scores may be aggregated within each instantiation type and then sorted, as they are all scalar values. Some threshold may be applied, and only types with scores above it may be retained, or only the lop N′ types retained. This is advantageous in that (a) this selection may be influenced by specific stimuli within each task type, and (b) this selection function itself may be optimized.

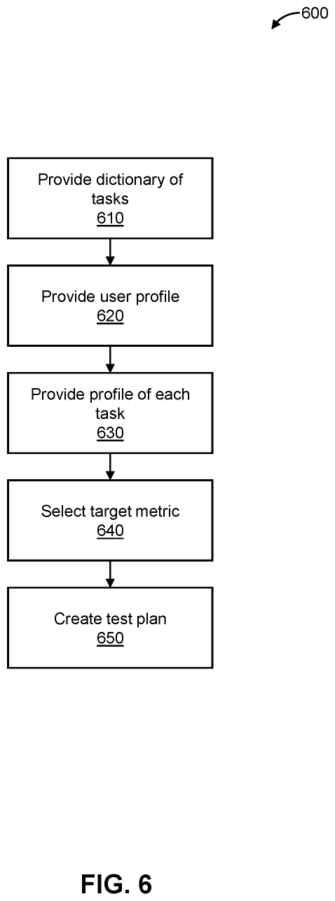

FIG. 6 illustrates a method for constructing an assessment plan for neurological and/or behavioral testing 600, in accordance with an embodiment. At block 610, plan constructor module 132 is provided with a dictionary or structure of available tasks. At block 620, plan constructor module 132 is provided with a user profile, the user profile being based in part on the prior performance of the subject being assessed in a subset of the available tasks. At block 630, plan constructor module 132 is provided with a profile of each task, in terms of what aspects of assessment that the task explores (e.g., the picture-naming task projects onto the dimensions of semantic memory, vision, word-finding, etc.) and its difficulty level across those aspects. At block 640, plan constructor module 132 is provided with a target metric that was selected for optimization, the selection being made by a real or virtual tester/clinician. At block 650, plan constructor module 132 creates a test plan based on the data or information generated or produced in the previous steps to produce a sequence of tasks meant to optimize the target metric. In other embodiments, the order of steps performed in the method may be changed, and some steps may be combined.

Plan constructor module 132 may employ method 600 to automatically construct an assessment plan for neurological and/or behavioral testing based on the subject's profile and diagnostic needs. Such a method may be useful for assigning an assessment plan to a subject engaged in cognitive, behavioral, psychological, or motor function assessment. An assessment consists of a set of tasks, each of which may evaluate different aspects of cognition (e.g., language production and comprehension, memory, visuospatial ability, etc.) and may have multiple task instances (i.e., task versions) of variable difficulty, where difficulty is defined relative to each subject based on their personal cognitive status. For example, picture description is an example of a task present in cognitive assessment, while the various pictures which may be shown to the subject as part of the task are examples of task instances with variable difficulty. The difficulty attribute of task instances is not an absolute characteristic of the instances, but rather depends on the subject performing the task (e.g., a person with frontotemporal lobar degeneration may experience difficulty talking about a picture depicting animate objects, while a healthy person would not). The assessment may output a continuous quantitative measure of cognitive, behavioral, psychological, or motor performance, and/or a discrete class indicating the diagnosis which is the most likely underlying cause of the detected symptoms (e.g., ‘Alzheimer's disease’, ‘Parkinson's disease’, ‘healthy’, etc.), and/or a continuous probability of each diagnosis (e.g., ‘55%—Alzheimer's disease; 40%—Mild cognitive impairment; 5%—healthy’).

In an embodiment, plan constructor module 132 may carry out method 600 using an artificial neural network (ANN). The ANN may consist of deep learning frameworks such as PyTorch, TensorFlow, or Keras.

In a further embodiment, plan constructor module 132 may carry out method 600 by utilizing a reward function that is set to specifically tease apart differences among clinically relevant categories (e.g., diseases). Subjects may exhibit a “ceiling effect” if the tasks in an assessment are too easy, especially for subjects with early signs of cognitive decline. An appropriate assessment plan in that scenario would ensure that the tasks became increasingly difficult, along relevant dimensions, in order to detect subtle signs of cognitive decline. In contrast to the “ceiling effect”, subjects with more advanced forms of cognitive impairment might exhibit the “floor effect” if they find that all subtasks are too difficult. Either the “floor effect” or “ceiling effect” would make detecting subtle cognitive issues difficult. Advantageously, task difficulty can be adjusted along relevant dimensions to detect the subject's level of impairment. Task difficulty level is automatically generated, after collecting demographic information on the individual. The information collected includes: age of subject, education level, and any diagnosed cognitive or psychiatric condition (if any).

In a further embodiment, plan constructor module 132 may carry out method 600 by utilizing a reward function that is set to provide easy tasks so that the subject continues to use the platform (e.g., to reduce their stress or optimize their sense of reward) and is able to complete the cognitive assessment each time. The cognitive assessment may consist of a number of tasks that are low stress/anxiety-provoking, such as the picture description and paragraph reading and recall tasks. Each assessment session may consist of one or more of the easy tasks: (i) at the beginning of the test session, to boost reward function; and (ii) after comparatively challenging tasks, to reduce any anxiety/stress due to task difficulty.

In a further embodiment, plan constructor module 132 may carry out method 600 in such a manner that the type of task changes (e.g., from picture description to fluency). The method may assess cognitive measures through a number of different types of tasks, such as picture description tasks, semantic and phonemic fluency tasks, and paragraph reading and recall task.

Picture description tasks is one type of task. Verbal response/description of a picture by the subject is recorded. Speech from the picture description is analyzed, and sub-scores for semantic memory, language use and comprehension (e.g., grammar/syntax, unique words, relevant answers), acoustic measures (e.g., speech duration, pauses), and thought process (coherence, information units, topic shifts) are computed for this task type.

Semantic and phonemic fluency tasks is another type of task. Speech is evaluated as with picture description tasks. However, the fluency tasks are more specific for assessing domains like: working memory, naming ability, semantic associations, and executive control.

Paragraph reading and recall tasks is another type of task. Again, speech is analyzed, but the main focus for this task type is to gauge natural tonal variations and accent of the subject being tested. Comparison of the subject in this task allows their acoustics to be compared to data pools (e.g., people with different accents, age-related tonal variations) in a database and determine if the subject has any acoustic impairment. In addition, this task serves as an easy, low-stress task (high-reward function) and is sometimes presented at the beginning of the assessment session. The delayed recall portion of this task tests memory acquisition, recall function, and language expression.

Variations in task type are flexible, unlike those of standard neuropsychological assessments. Standard tasks have a rigid task order, which makes it challenging to identify and investigate impairments in specific cognitive domains. To avoid this problem, tasks can be presented in any order, depending on the reward/cost functions. The option for task selection allows administrators (e.g., clinicians) to focus on evaluating performance in a subject's impaired cognitive domain, such as language expression.

Alternatively, a sequence of tasks for a particular session can be predetermined (e.g., in a research setting), allowing for even distribution of tasks of different types or with different levels of difficulty. This may help reduce directed attention fatigue seen in standard tests, where, for instance, subjects complete all attention-related tasks at a time.

In a further embodiment, plan constructor module 132 may carry out method 600 in such a manner that the stimuli within a task changes (e.g., between specific pictures) using information about those stimuli. In general, the method of changing the stimuli for a particular task (by using a large bank of automatically generated task instances) assists in conducting multiple longitudinal assessments and can help prevent learning effects over time. The method advantageously enables more frequent monitoring of cognitive status in elderly adult subjects who show early signs of cognitive decline, allowing healthcare professionals and caregivers to provide appropriate intervention and care. Furthermore, early identification of the preclinical stages of a cognitive disorder assists in studying disease pathology and facilitating the discovery of treatments, as suggested in recommendations from associations for various neuropsychiatric conditions, such as the Alzheimer's Association workgroups. Variations of a task stimulus within a specific session and/or longitudinally (across multiple sessions) include: picture description task, semantic fluency task, phonemic fluency task, and paragraph reading.

Picture description tasks can be varied. A different picture stimulus is presented each time, even for longitudinal sessions. Variants may include a non-personal photograph of a daily-life scenario; this mimics a real-life, low-stress task (e.g., describing a photo). The task may utilize non-personal photographs to avoid emotional distress for subjects with cognitive deficits who may be unable to recall personal memories. Another variant may include a line drawing picture; this is a standard stimulus type for a picture description task (containing sufficient details for description). Collecting within-subject data for different picture description stimuli may help: (i) account for daily fluctuations in performance and help prevent false positives (e.g., faulty diagnosis of disease progression), especially in cases of longitudinal assessments; (ii) select preferred stimulus (e.g., examiner may choose a particular type of picture task to further test a subject's specific condition).

Semantic fluency tasks can be varied. These assess semantic memory for categorical objects. Each time, a unique semantic category task may be presented. Examples of stimulus variants include categories such as: “animal”, “food”, and “household object”. The different categories allow investigation of a subject's semantic associations for words, as well as accessibility of semantic and working memory. Command of semantic associations may also help inform the specific subtype of cognitive disorder that a subject has.

Phonemic fluency tasks can be varied. These assess word recall/vocabulary and phonological function. Each time, a unique phoneme stimulus can be presented. Examples of stimulus variants include letters such as T, ‘a’, and ‘s’. The different (but equivalent) stimulus variants assess memory function and check for the presence of phonological errors (indicative of specific stages or subtypes of cognitive/language impairment).

Paragraph reading can be varied. A different paragraph can be presented for each consecutive assessment. The paragraph variants test the subject's accent and tonal variations for different words, with different phonemes.

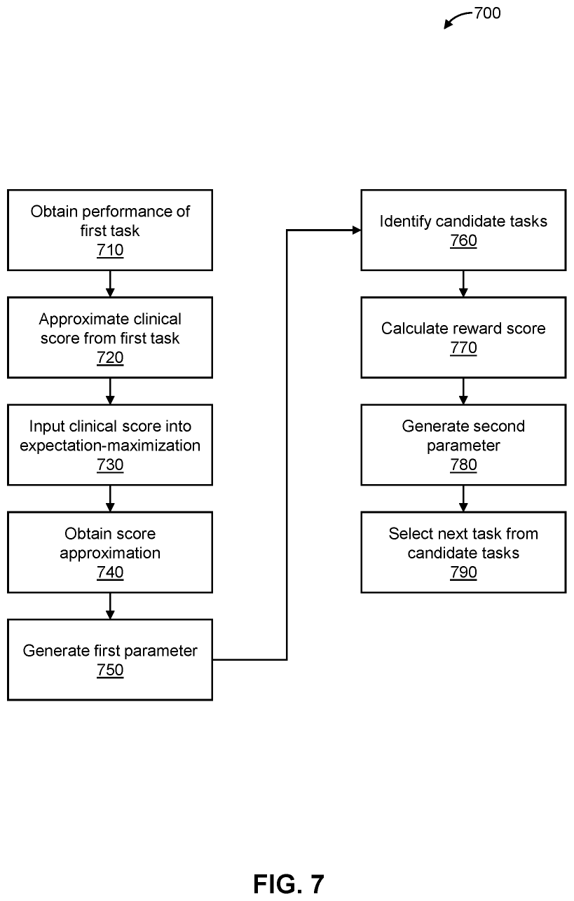

FIG. 7 illustrates a method of dynamically determining a next task in a cognitive assessment 700, in accordance with an embodiment. At block 710, a system configured to perform the method (e.g., system 100) obtains performance measurements of a first task. At block 720, the system approximates a clinical score from the performance measurements of the first task. At block 730, the system inputs the clinical score into an expectation-maximization function. At block 740, the system obtains a score approximation from the expectation-maximization function. At block 750, the system generates a first parameter based on the score approximation and a target metric. At block 760, the system identifies candidate tasks based on the first parameter and the target metric. At block 770, the system calculates a reward score based on the candidate task and the first parameter for each of the candidate tasks. At block 780, the system generates a second parameter based on the reward score and the first parameter. At block 790, the system selects the next task from the candidate tasks that maximizes the target metric.

Although the invention has been described with reference to certain specific embodiments, various modifications thereof will be apparent to those skilled in the art without departing from the spirit and scope of the invention as outlined in the claims appended hereto.

1. A system for scoring language tasks for assessment of cognition, the system comprising a processing unit and a data storage, the data storage configured to store a plurality of instructions which when executed by the processing unit cause the processing unit to execute:
a collector configured to receive language task data, the language task data comprising at least one of speech, text, and multiple-choice selections obtained from a user;
an extractor configured to extract a plurality of language features from the received language task data using an automated language processing technique, the plurality of language features comprising at least one of an acoustic measure, a lexicosyntactic measure, and a semantic measure; and
a score producer configured to use the extracted plurality of language features to automatically produce a plurality of scores, the plurality of scores generated using an automated language processing algorithm.

2. The system of claim 1, wherein the acoustic measure comprises at least one of Mel-frequency cepstral coefficients (MFCCs), jitter and shimmer measures, aperiodicity features, measures of signal-to-noise ratio, pauses, and fillers.

3. The system of claim 1, wherein the lexicosyntactic measure is extracted from textual responses and transcriptions of verbal responses, and comprise at least one of frequency of production rules, phrase types, and word types, length measures, frequency of use of passive voice and subordination or coordination, and syntactic complexity.

4. The system of claim 1, wherein the semantic measure is extracted by comparing subject responses to ground truth responses for each language task, the ground truth comprises at least one of a count, an arithmetic mean, or a sum of boxes.

5. The system of claim 1, wherein the automated language processing algorithms comprise at least one of models for semantic similarity among words or larger passages, computation of distance between vector representations of words or larger passages, traversal of graph-based representations of lexical and linguistic relations, computation of lexical cohesiveness and coherence, topic identification, and summarizing techniques.

6. The system of claim 5, wherein the automated language processing algorithms further comprise a confidence value for each score.

7. The system of claim 1, wherein the language tasks comprise at least one of vocabulary assessment through word definition, image naming, picture description, sentence completion or re-ordering, story recall, Winograd schema problems, phrase re-ordering, random item generation, color naming with Stroop interference, and self-assessed general disposition.

8. The system of claim 1, wherein the automated language processing algorithm comprises a machine learning model to take the language features as an input dataset and output the plurality of scores, the machine learning model is trained using labels for the output scores received from a subset of human interpreters.

9. The system of claim 8 wherein the machine learning model comprises a first aggregation function that provides scores based on a relationship between the language features and the labels and a second aggregation function between pairs of language features, the machine learning model applies hierarchical clustering to obtain a graph structure over the language features using the second aggregation function as a distance metric, the machine learning model determines a node from the graph structure that is most representative of at least one of the plurality of scores by sorting nodes according to relevance scores and selects the top-ranking node, the machine learning model returns the value of the first aggregation function as applied to the top-ranking node.

10. The system of claim 9, wherein the relevance score to each node within the graph structure is determined using the first aggregation function as a relevance metric.

11. A computer-implemented method for scoring language tasks for assessment of cognition, the method comprises:
receiving language task data, the language task data comprising at least one of speech, text, and multiple-choice selections obtained from a user;
extracting a plurality of language features from the received language task data using an automated language processing technique, the plurality of language features comprising at least one of an acoustic measure, a lexicosyntactic measure, and a semantic measure; and
using the extracted plurality of language features to automatically produce a plurality of scores, the plurality of scores generated using an automated language processing algorithm.

12. The method of claim 11, wherein the acoustic measure comprises at least one of Mel-frequency cepstral coefficients (MFCCs), jitter and shimmer measures, aperiodicity features, measures of signal-to-noise ratio, pauses, and fillers.

13. The method of claim 11, wherein the lexicosyntactic measure is extracted from textual responses and transcriptions of verbal responses, and comprise at least one of frequency of production rules, phrase types, and word types, length measures, frequency of use of passive voice and subordination or coordination, and syntactic complexity.

14. The method of claim 11, wherein the semantic measure is extracted by comparing subject responses to ground truth responses for each language task, the ground truth comprises at least one of a count, an arithmetic mean, or a sum of boxes.

15. The method of claim 11, wherein the automated language processing algorithms comprise at least one of models for semantic similarity among words or larger passages, computation of distance between vector representations of words or larger passages, traversal of graph-based representations of lexical and linguistic relations, computation of lexical cohesiveness and coherence, topic identification, and summarizing techniques.

16. The method of claim 15, wherein the automated language processing algorithms further comprise a confidence value for each score.

17. The method of claim 11, wherein the language tasks comprise at least one of vocabulary assessment through word definition, image naming, picture description, sentence completion or re-ordering, story recall, Winograd schema problems, phrase re-ordering, random item generation, color naming with Stroop interference, and self-assessed general disposition.

18. The method of claim 11, wherein the automated language processing algorithm comprises a machine learning model to take the language features as an input dataset and output the plurality of scores, the machine learning model is trained using labels for the output scores received from a subset of human interpreters.

19. The method of claim 18, wherein the machine learning model comprises a first aggregation function that provides scores based on a relationship between the language features and the labels and a second aggregation function between pairs of language features, the machine learning model applies hierarchical clustering to obtain a graph structure over the language features using the second aggregation function as a distance metric, the machine learning model determines a node from the graph structure that is most representative of at least one of the plurality of scores by sorting nodes according to relevance scores and selects the top-ranking node, the machine learning model returns the value of the first aggregation function as applied to the top-ranking node.

20. The method of claim 19, wherein the relevance score to each node within the graph structure is determined using the first aggregation function as a relevance metric.
